(19) United States
(12) Patent Application Publication
(10) Pub. No.: US 2024/0335170 A1
(43) Pub. Date: Oct. 10, 2024

(54) SYSTEM AND METHOD FOR ELECTROCARDIOGRAM IMAGE-BASED PATIENT EVALUATION

(71) Applicant: Seoul National University Hospital, Seoul (KR)

(72) Inventor: Joonghee Kim, Seongnam-Si (KR)

(73) Assignee: Seoul National University Hospital, Seoul (KR)

(21) Appl. No.: 18/287,018

(22) PCT Filed: Apr. 15, 2022
(86) PCT No.: PCT/KR2022/005488

§ 371 Date: May 8, 2024

(30) Foreign Application Priority Data
Apr. 16, 2021 (KR) 10-2021-0049894
Nov. 22, 2021 (KR) 10-2021-0161788

(51) Int. Cl. A61B 5/00; A61B 5/358; A61B 5/36; A61B 5/361; A61B 5/363; A61B 5/364; G16H 10/60; G16H 15/00; G16H 20/00; G16H 50/70

(52) U.S. Cl. CPC A61B 5/7267; A61B 5/358; A61B 5/36; A61B 5/361; A61B 5/363; A61B 5/364; A61B 5/7275; A61B 5/743; A61B 5/7435; G16H 10/60; G16H 15/00; G16H 20/00; G16H 50/70; A61B 2560/045

(57) ABSTRACT

Embodiments relate to a system and method for electrocardiogram image-based patient evaluation, wherein: a source electrocardiogram image of a target patient is acquired by a user terminal; a server is requested by the user terminal to evaluate the target patient, the request including the source electrocardiogram image; electrocardiogram image-based evaluation information of the patient is generated by the server, the evaluation information of the target patient is transmitted to the user terminal; and reported feedback based on the evaluation information of the target patient is provided by the user terminal.

### TECHNICAL FIELD

Embodiments of the present application relate to technology for evaluating a patient's state, and more particularly, to a system and method for evaluating a patient's state, including arrhythmia, based on an electrocardiogram image, and displaying evaluation information.

### BACKGROUND ART

As domestic economies and living environments have become more westernized, heart disease has become one of the leading causes of death in Korea, along with cancer and cerebrovascular disease. The most important signal for the pre-diagnosis and prognosis of these heart diseases is an electrocardiogram (ECG) signal generated by the heart's activity.

In general hospitals, an electrocardiogram is performed on every patient who visits the hospital for about two to three minutes to monitor the patient's state, so that it is possible to diagnose whether there are any abnormalities in the heart. In this case, medical professionals analyze the waveform of the electrocardiogram signal to diagnose whether a patient has a heart disease.

However, it is difficult for even the most experienced medical professionals to diagnose a specific heart disease that a patient may have from a variety of heart diseases based on the electrocardiogram signal alone. This is because in the electrocardiogram signal with similar waveforms repeated at low intensity, it is necessary to accurately analyze a variety of heart diseases that can be categorized into numerous specific detailed diseases, such as vascular related emergency disease groups such as coronary artery disease (CAD), electrolyte abnormalities, shock, pulmonary edema, respiratory failure, and myocarditis; cardiac dysfunction disease groups such as ventricular failure and valvular heart abnormalities; and arrhythmia.

There are attempts to use computerized devices to analyze the specific detailed diseases, such as whether a patient has a specific arrhythmia, but there are limitations in terms of accuracy in simultaneously judging other heart diseases in the patient with arrhythmia. The electrocardiogram signal of the patient with arrhythmia is already affected by the arrhythmia symptoms, such as irregular heart rhythm or abnormal heart rate, and is implemented as an abnormal waveform, which makes it difficult to analyze whether the abnormal waveform is caused by the arrhythmia or another heart disease.

### DOCUMENTS OF RELATED ART

### Patent Documents

(Patent Document 1) Korean Patent Application Laid-Open No. 10-2014-0063100 (May 27, 2014)

### DISCLOSURE

### Technical Problem

According to various aspects of the present application, it is an object to provide a system and method for patient evaluation for evaluating whether a patient has various heart diseases, including arrhythmias, based on an electrocardiogram image and generating a reporting screen displaying evaluation information, and a computer-readable recording medium for recording the same.

### Technical Solution

A user terminal for patient evaluation based on an electrocardiogram image, according to one aspect of the present application, includes a processor, and a photographing unit. The user terminal may be configured to acquire a source electrocardiogram image of a target patient, transmit a request including the source electrocardiogram image to a server, receive evaluation information of the target patient from the server, and provide reported feedback based on the evaluation information. The evaluation information includes one or more of first diagnostic information including an electrocardiogram image and a result of evaluation of a heart rhythm of the target patient. The reported feedback includes one or more of a first region displaying the electrocardiogram image and a second region displaying the first diagnostic information.

In an embodiment, the user terminal further includes a display unit, and the configuration of providing the reported feedback may include displaying a reporting screen using the display unit.

In an embodiment, the evaluation information may further include second diagnostic information including an evaluation value of the target patient for an item in a category different from a heart rhythm category, as a result of evaluation of a different aspect of the heart rhythm of the target patient. The reporting screen further includes a third region displaying the first diagnostic information and/or the second diagnostic information in a graph.

In an embodiment, in the third region, an x-axis of the graph may represent at least some of evaluated items and a y-axis may represent an evaluation value, for example, an evaluation value of the target patient for the corresponding item, in which an evaluation value for each item may be expressed as a pointer.

In an embodiment, the evaluation information may further include distribution information on the evaluation value for an item. The reporting screen is further configured such that the distribution information of the evaluation value for the item is represented as a sub-region on the third region in which a pointer of the evaluation value is positioned.

In an embodiment, the sub-region may have a length of shape according to a confidence interval of distribution of each evaluation value.

In an embodiment, the first diagnostic information is calculated by selecting a normal rhythm, any one of a first group of arrhythmia types, or any one of a second group of arrhythmia types. When the heart rhythm of the target patient in the first diagnostic information is the second group of arrhythmia types, it is configured that the reported feedback including a result of evaluation consisting only of the electrocardiogram image and the second diagnostic information is provided.

In an embodiment, the first group of arrhythmia types may include one or more of atrial flutter, PSVT, atrial tachycardia, ventricular tachycardia, and a pacemaker. The second group of arrhythmia types consists of some or all of the remaining arrhythmia types that do not belong to the first group among all of the plurality of pre-specified arrhythmia types.

In an embodiment, the user terminal may further include a photographing unit, in which the user terminal may be further configured to select a plurality of reference points for determining a frame guideline in a photographed image, and adjust a size of an electrocardiogram signal region in the photographed image to fit the determined frame guideline, in order to acquire the source electrocardiogram image.

In an embodiment, the determined frame guideline includes an electrocardiogram signal region, and in which at least some of the selected plurality of reference points are positioned inside the frame guideline.

In an embodiment, the user terminal may be further configured to remove a region displaying personal information of a patient to anonymize the source electrocardiogram image.

In an embodiment, the user terminal may be further configured to provide the result of the evaluation to an expert account registered in a database.

In an embodiment, the user terminal may be configured to provide the user terminal with feedback from the expert account on the result of the evaluation.

In an embodiment, the result of the evaluation provided to the expert account may be provided in a manner that includes at least one of text message, email, or push notification.

In an embodiment, the feedback from the expert account provided to the user terminal may be in the form of at least one of an image, a voice, or a text.

In an embodiment, the user terminal may be configured to provide the result of the evaluation to a medical professional or a hospital account registered in the database.

In an embodiment, the result of the evaluation provided to the medical professional or hospital account may be provided in a manner that includes at least one of text message, email, or push notification.

In an embodiment, the user terminal may be configured to further provide the medical professional or hospital account registered in the database with a list of preparations based on the result of the evaluation.

In an embodiment, the user terminal may be configured to receive feedback from the medical professional or hospital account on the result of the evaluation and provide the feedback to the user terminal.

In an embodiment, the feedback from the medical professional or hospital account provided to the user terminal may be in the form of at least one of an image, a voice, or a text.

There is provided a server in communication with a user terminal for patient evaluation based on an electrocardiogram image, according to another aspect of the present application, the server may be configured to receive, from the user terminal, a source electrocardiogram image of a target patient including an electrocardiogram signal region, and generate evaluation information of the target patient based on the source electrocardiogram image. The evaluation information includes one or more of first diagnostic information including an electrocardiogram image and a result of evaluation of a heart rhythm of the target patient, and second diagnostic information including an evaluation value of the target patient for an item in a category different from a heart rhythm category.

In an embodiment, the server may be configured to extract waveform data of an electrocardiogram signal from the source electrocardiogram image, and generate evaluation information of the target patient based on the extracted waveform data, in order to generate the evaluation information of the target patient. For example, waveform data may be extracted from the source electrocardiogram image, the waveform data may be transformed into one-dimensional data, for example, one-dimensional multi-channel data using, for example, a neural network, and then evaluation information may be generated based on the transformed data. In an embodiment, the server may include a first artificial neural network pre-trained to extract waveform data of a signal from an input image, and a second artificial neural network pre-trained to input the waveform data to calculate an evaluation value of the target patient for a pre-specified item. The first artificial neural network is modeled as a two-dimensional (2D)-CNN, a visual transformer (VIT), or a multilayer perceptron (MLP) structure, and the second artificial neural network is modeled as a one-dimensional (1D)-CNN structure.

In an embodiment, the server may directly process two-dimensional data to generate evaluation information waveform from the source electrocardiogram image. For example, a third artificial neural network pre-trained to input the source electrocardiogram image to calculate the evaluation value of the target patient for the pre-specified item may be included. The third artificial neural network is modeled as a two-dimensional (2D)-CNN, a visual transformer (VIT), or a multilayer perceptron (MLP) structure.

In the embodiments described above, the second artificial neural network or third artificial neural network is modeled to have a multi-label output stage, and calculates an absolute evaluation value that individually represents the likelihood of corresponding to each of the pre-specified items.

In the embodiments described above, the second artificial neural network or third artificial neural network is modeled to have a multi-label output stage, and has a probability distribution relationship such that a sum of the evaluation values for each item calculated is equal to 1.

In the embodiments described above, the server may be further configured to calculate distribution information for an evaluation value for each item. The distribution information is based on specific distribution formed by an evaluation value for an item at each sequence, which is calculated by repeating the same evaluation operation with the same input value, or based on specific distribution formed by an evaluation value for an item at each sequence, which is calculated by performing an evaluation operation with a transformed input value in which an augmentation technique used in training of the second artificial neural network or the third artificial neural network is applied to the same input value.

In the embodiments described above, the server may determine a type of heart rhythm of the target patient through the second artificial neural network or the third artificial neural network, and when the heart rhythm of the target patient is a first group of arrhythmia types, the server may partially perform or may not perform evaluation for an item in a category different from the heart rhythm, and may generate evaluation information including a result of evaluation for the heart rhythm of the target patient, or a result of evaluation for the electrocardiogram image and the heart rhythm of the target patient.

In an embodiment, the server may crop an electrocardiogram signal region from the source electrocardiogram image, generate a transformed image in which at least one of a view, a size, or a shape is transformed by processing the cropped image with a perspective transformation, calculate an original aspect ratio of the electrocardiogram signal region in the source electrocardiogram signal, correct an aspect ratio of the transformed image to the original aspect ratio to generate an electrocardiogram signal patch, and generate evaluation information of the target patient based on the electrocardiogram signal patch.

In an embodiment, the server may provide the result of the evaluation to an expert account registered in a database.

In an embodiment, the server may provide the user terminal with feedback from the expert account on the result of the evaluation.

In an embodiment, the result of the evaluation provided to the expert account may include at least one of text message, email, or push notification.

In an embodiment, the feedback from the expert account provided to the user terminal may be in the form of at least one of an image, a voice, or a text.

In an embodiment, the server may provide the result of the evaluation to a medical professional or hospital account registered in the database.

In an embodiment, the result of the evaluation provided to the medical professional or hospital account may be provided in a manner that includes at least one of text message, email, or push notification.

In an embodiment, the server may further provide the medical professional or hospital account registered in the database with a list of preparations based on the result of the evaluation.

In an embodiment, the server may receive feedback from the medical professional or hospital account on the result of the evaluation and provide the feedback to the user terminal.

In an embodiment, the feedback from the medical professional or hospital account provided to the user terminal may be in the form of at least one of an image, a voice, or a text.

There is provided a method of evaluating a patient based on an electrocardiogram image according to still another aspect of the present application, the method may include: acquiring, by a user terminal, a source electrocardiogram image of a target patient; requesting, by the user terminal, evaluation of the target patient to a server—in which the request includes the source electrocardiogram image; generating, by the server, evaluation information of the target patient based on an electrocardiogram image; transmitting the evaluation information of the target patient to the user terminal; and providing, by the user terminal, reported feedback based on the evaluation information of the target patient.

A computer-readable recording medium according to yet another aspect of the present application, when executed by a processor, may record a program for performing the method of evaluating a patient based on an electrocardiogram image according to the embodiments described above.

### Advantageous Effects

The system for electrocardiogram image-based patient evaluation, according to one aspect of the present application, can also evaluate a state of a target patient in an aspect of heart rhythm, such as arrhythmia, and in a different aspect, such as another heart disease/state event, based on an electrocardiogram image.

Further, the system for electrocardiogram image-based patient evaluation can also provide a user with a reporting screen configured such that the user can easily recognize visually results of the evaluation in various aspects.

The effects of the present application are not limited to those mentioned above, and other effects not mentioned will be clearly understood by those skilled in the art from the description of the claims.

### DESCRIPTION OF DRAWINGS

To more clearly describe the technical solutions of the embodiments of the present application or the related art, the drawings required in the description of the embodiments are briefly described below. It should be understood that the flowing drawings are intended to explain the embodiments of the present specification but not to limit the embodiments of the present specification. In addition, for clarity of descriptions, some elements may be illustrated in the drawings below with various variations, including exaggeration and omission.

FIG. 1 is a schematic view of an operation of a system for electrocardiogram image-based patient evaluation, according to one aspect of the present application.

FIG. 2 is a tabular view illustrating evaluation information on a first analysis group, according to an embodiment of the present application.

FIG. 3 is a view illustrating a reporting screen including an electrocardiogram image and analysis results, according to an embodiment of the present application.

FIG. 4 is a view illustrating a screen summarizing evaluation information, according to an embodiment of the present application.

FIG. 5 illustrates an interface screen for inputting auxiliary information on a target patient, according to an embodiment of the present application.

FIG. 6 is a view illustrating a first matching screen displaying a cluster of matching patients, according to an embodiment of the present application.

FIG. 7 is a view illustrating a second matching screen for calculating evaluation information on a single patient over time, according to an embodiment of the present application.

FIG. 8 is a flowchart of a method of electrocardiogram image-based patient evaluation, according to an embodiment of the present application.

FIGS. 9A to 9C are views illustrating a process of acquiring a source electrocardiogram image according to a frame guideline, according to an embodiment of the present application.

FIG. 10 is a view illustrating a process of acquiring a source electrocardiogram image according to a frame guideline, according to another embodiment of the present application.

FIG. 11 illustrates an interface screen for anonymizing an electrocardiogram image, according to an embodiment of the present application.

### MODE FOR DISCLOSURE

Embodiments will be described with reference to the accompanying drawings, but the principles disclosed herein may be implemented in various different forms and should not be considered limited to the embodiments described herein. In the detailed description of the invention, detailed descriptions of well-known features and technologies may be omitted to avoid making features of the embodiments unnecessarily obscure.

The technical terms used herein are merely for the purpose of describing a specific exemplary embodiment, and not intended to limit the present application. Singular expressions used herein include plural expressions unless they have definitely opposite meanings. The terms “comprises” and/or “comprising” used in the specification specify particular features, regions, integers, steps, operations, elements, and/or components, but do not exclude the presence or addition of other features, regions integers, steps, operations, elements, and/or components.

Unless otherwise defined, all terms used herein including technical or scientific terms have the same meanings as meanings which are generally understood by those skilled in the art to which the present application belongs. It shall be additionally construed that terms, which are defined in dictionaries generally used, have meanings matching the related art document and currently disclosed contents, and the terms shall not be construed as ideal or excessively formal meanings unless clearly defined in the present application.

Hereinafter, embodiments of the present application will be described in detail with reference to the drawings.

### System Architecture

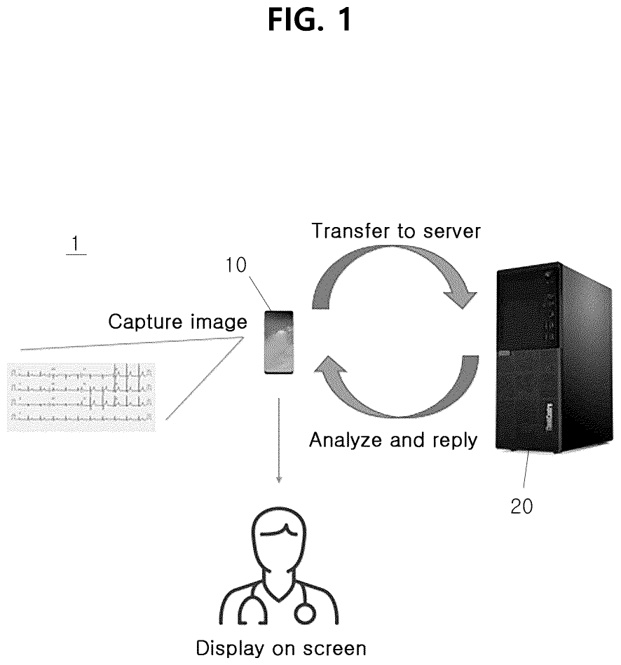

FIG. 1 is a schematic view of an operation of a system for electrocardiogram image-based patient evaluation, according to one aspect of the present application.

With reference to FIG. 1, a system 1 for electrocardiogram image-based patient evaluation includes a user terminal 10 and a server 20.

The system 1 for electrocardiogram image-based patient evaluation according to embodiments may have an aspect that is entirely hardware, entirely software, or partly hardware and partly software. For example, the system may refer to hardware having data processing capabilities and operating software to drive the hardware. In the present specification, terms such as “unit,” “module,” “device,” or “system” are intended to refer not only to hardware, but also to combinations of software driven by the corresponding hardware. For example, hardware can be a data processing device that includes a central processing unit (CPU), a graphics processing unit (GPU), or another processor. In addition, software can refer to a running process, an object, an executable, a thread of execution, a program, and the like.

The user terminal 10 may be any kind of terminal capable of data communication. The user terminal 10 includes a processor and may be a wireless terminal (e.g., a cell phone, a personal digital assistant (PDA), a laptop, a smart phone, or the like) that wirelessly connects to a data network (e.g., the Internet), or a wired terminal (e.g., a PC, a laptop, a kiosk, or the like) that wiredly connects to a data network.

The user terminal 10 acquires an electrocardiogram image of a target patient for evaluation. The electrocardiogram image includes a region representing an electrocardiogram signal. The electrocardiogram image may include a plurality of waveforms representing an electrocardiogram signal.

The electrocardiogram image relies on an electrocardiogram measuring device. When the electrocardiogram measuring device has 12 leads, the electrocardiogram image may include a waveform of an electrocardiogram signal for each lead.

The user terminal 10 may also include a photographing unit (not illustrated) for recording an electrocardiogram image. The photographing unit may also include a camera, an image sensor, or other unit capable of recording a waveform of the electrocardiogram signal.

The user terminal 10 may also acquire a source electrocardiogram image by photographing an object (e.g., a printout, a display device) that displays the electrocardiogram signal on a surface thereof. A section of the electrocardiogram signal in the source electrocardiogram image depends on a photographic field of view.

As described above, the electrocardiogram image used in the system 1 for patient evaluation is not limited to an image including only the electrocardiogram signal region, but also may be an image including the electrocardiogram signal region and other regions.

In addition, the user terminal 10 may receive the electrocardiogram image of the target patient from an external device. The user terminal 10 may also receive electrocardiogram image data photographed or pre-stored by an external device through wired or wireless telecommunication. The user terminal 10 may also transmit information on the patient targeted for evaluation to the server 20. The user terminal 10 may also receive evaluation information on the target patient.

The user terminal 10 may be any kind of terminal capable of data communication, so as to be able to communicate with the server 20 or an external device. For example, the user terminal 10 includes a processor and may be a wireless terminal (e.g., a cell phone, a personal digital assistant (PDA), a laptop, a smart phone, or the like) that wirelessly connects to a data network (e.g., the Internet), or a wired terminal (e.g., a PC, a laptop, an ATM, or the like) that wiredly connects to a data network.

In addition, the user terminal 10 may store the electrocardiogram image of the target patient. The user terminal 10 may further store information associated with the electrocardiogram image of the target patient. The information associated with the electrocardiogram image may include information on the target patient and/or evaluation information.

The information on the patient includes patient's personal information. The patient's personal information may include the patient's name, social security number, gender, age, address, etc.

The user terminal 10 is configured to provide the evaluation information on the target patient to a user. In specific embodiments, the user terminal 10 may also display the evaluation information. To this end, the user terminal 10 may include a display unit (not illustrated) that displays information.

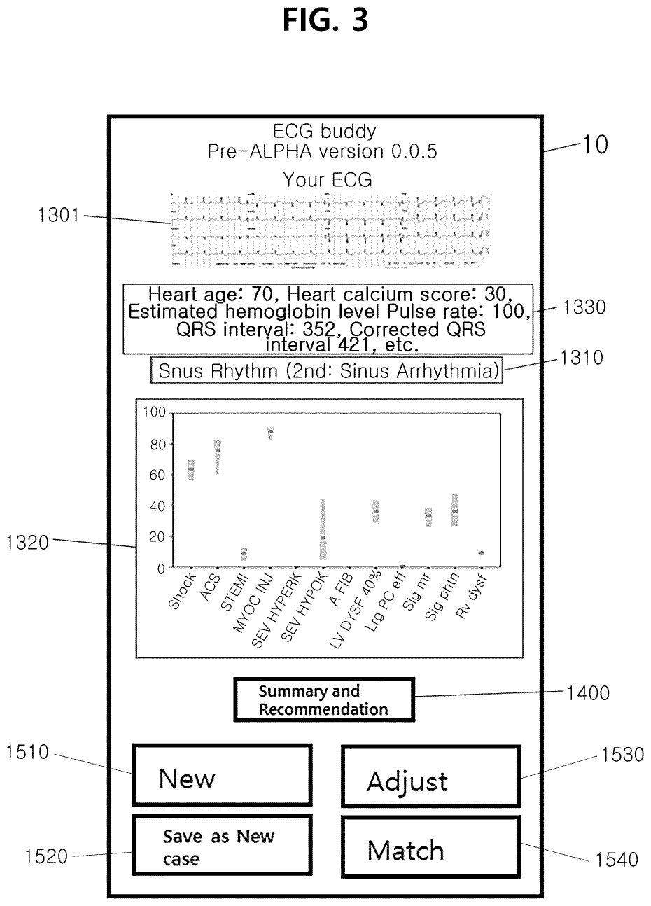

An operation of the user terminal 10 will be described in more detail with reference to FIGS. 3, 8, and 11 below.

The server 20 evaluates a state of the target patient based on the electrocardiogram image, and generates the evaluation information of the target patient. The evaluation information is a result of analyzing the electrocardiogram image and includes information that evaluates the state of the target patient from the perspective of the heart.

The server 20 is a plurality of computer systems or computer software implemented as a network server, configured to receive data (e.g., image data) from the user terminal 10 through a wired or wireless network. Here, a network server means a computer system and computer software (network server program) connected with subordinate devices capable of communicating with other network servers over a computer network, such as a private intranet or the Internet, that receives requests to perform tasks, performs the tasks, and provides results of the performance. However, in addition to these network server programs, it should be understood as a broader concept that includes a series of applications that run on the network server and, in some cases, various databases built therein.

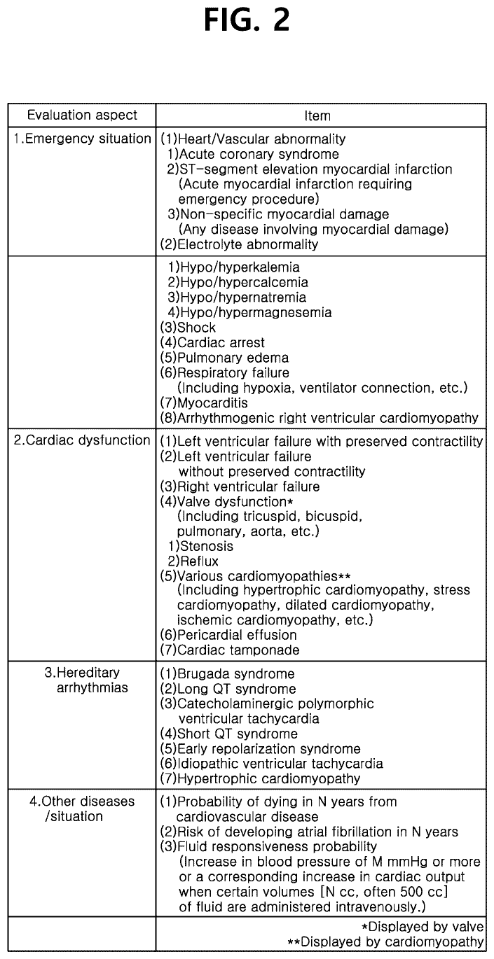

The user terminal 10 and server 20 may have software (application) installed and executed for performing a patient evaluation operation based on the electrocardiogram image, and the configuration of the user terminal 10 and server 20 may be controlled by the installed software. The patient evaluation operation based on the electrocardiogram image, which is performed at least in part by the user terminal 10 and the server 20, respectively, is described in more detail with reference to FIGS. 2 to 11 below.

### Patient Evaluation Based on Electrocardiogram Image

The server 20 may also calculate evaluation information that describes the evaluation of the state of the patient based on the electrocardiogram image received from the user terminal 10. The server 20 may process the electrocardiogram image data and generate the evaluation information by determining, based on the processed data, whether the state of the patient corresponds to at least one of the pre-stored items.

Evaluating the state of the patient includes determining whether the patient has a specific heart disease, whether the patient's biometric information (e.g., an electrolyte concentration) may cause a serious state event equivalent to the heart disease, and predicting the state of the patient in the future. To this end, the server 20 analyzes the electrocardiogram image of the target patient from various aspects.

The server 20 may also evaluate (or determine) the state of the target patient by analyzing the electrocardiogram image of the target patient in an aspect of heart rhythm, such as arrhythmia, in an aspect of emergency, in an aspect of a heart function, or in an aspect of other diseases/situations. The evaluation information from the server 20 is used to generate a reporting screen in FIG. 3 below.

In an embodiment, the server 20 may also evaluate the target patient in an aspect of rhythm. The items included in the evaluation information may also include types of heart rhythms.

The types of heart rhythms may include a normal sinus rhythm or a plurality of arrhythmia types. The plurality of arrhythmia types may also include numerous arrhythmia rhythms, including, for example, sinus bradycardia, second degree type I atrioventricular block (Mobitz I, or second degree type I heart block, referred to as Wenckebach), ventricular fibrillation, and polymorphic ventricular tachycardia (PVT), ventricular tachycardia, third degree heart block, first degree heart block, nodal rhythm, atrial flutter, atrial fibrillation, second degree type II, sinus rhythm with ST elevation, sinus tachycardia with unifocal PVC's and couplets, and multifocal atrial tachycardia (MAT), paroxysmal supraventricular tachycardia (PSVT), sinus rhythm with ventricular bigeminy, atrial tachycardia, junctional rhythm, and electronic pacemaker.

The server 20 may also determine, based on the electrocardiogram image or electrocardiogram signal of the target patient, the type of heart rhythm to which the target patient corresponds. The server 20 may also store a heart rhythm category consisting of a plurality of items, each indicating a type of heart rhythm. This category may have a plurality of items referring to each of the plurality of arrhythmia types described above.

The server 20 may determine the heart rhythm of the target patient to be a normal rhythm when an analysis result of the electrocardiogram image is calculated to correspond to an item indicating a normal rhythm.

Alternatively, the server 20 may determine that the heart rhythm of the target patient is a corresponding arrhythmia type from a pre-specified plurality of arrhythmia types when the heart of the target patient is unable to be categorized as a normal rhythm.

In addition, the server 20 may be further configured to evaluate the state of the target patient in a different aspect from the heart rhythm.

That is, for the same target patient, the server 20 may, in addition to determining the type of heart rhythm, such as arrhythmia, determine whether the target patient corresponds to an item in a different aspect.

In addition, the server 20 may be further configured to provide the result of the evaluation to an expert account registered in a database. An expert is a professional who can interpret or make further judgments about the result of the evaluation and provide feedback, including a medical professional, but is not limited thereto. The expert account may be a group account to which one or more experts belong. The server 20 provides the user terminal with feedback from the expert account on the result of the evaluation, in which the feedback from the expert account includes an interpretation or further judgment of the expert or expert group on the result of the evaluation. The result of the evaluation provided to the expert account may be provided in a manner that includes at least one of text message, email, or push notification. The feedback from the expert account provided to the user terminal may be in the form of at least one of an image, a voice, or a text.

Additionally, the server 20 may be configured to provide the result of the evaluation to a medical professional or hospital account registered in the database. The medical professional or hospital may be used such that, for example, in an emergency situation, a patient or a first aid performer, such as an emergency responder, may provide the result of the evaluation to a hospital emergency room, related specialty department, or medical professional, preferably in advance of the patient's arrival, such that the medical professional or hospital may be prepared in advance, but is not limited thereto. The result of the evaluation provided to the medical professional or hospital account may be provided in a manner that includes at least one of text message, email, or push notification.

The server 20 may be further configured to provide the medical professional or hospital account registered in the database with a list of preparations based on the result of the evaluation, which may include, for example, a list of preparations in the event of an emergency situation as described above. The server may receive feedback from the medical professional or hospital account on the result of the evaluation and provide the feedback to the user terminal. The feedback from the medical professional or hospital account may include an interpretation, further judgment, or instructions regarding the result of the evaluation. For example, the feedback may include the interpretation, further judgment regarding the result of the evaluation, or instructions from the medical professional, but is not limited thereto. The feedback from the medical professional or hospital account provided to the user terminal may be in the form of at least one of an image, a voice, or a text.

FIG. 3 is a view of an evaluation table illustrating items of various aspects, according to an embodiment of the present application.

With reference to FIG. 3, the items of the different aspects may include a heart disease associated with a cardiac dysfunction; a state event or disease associated with an emergency situation; and/or other state events/diseases. The server 20 may store a cardiac dysfunction category of one or more items; an emergency situation category of one or more items; and/or other state event/disease categories.

The emergency situation category consists of items where the state of the target patient indicates a disease or state that may be diagnosed as an emergency situation. The emergency situation category may be categorized into multiple groups. Each group may also consist of at least one item.

In an embodiment, the emergency situation category may be categorized into one or more groups of: a cardiac/vascular abnormality group; an electrolyte abnormality group; a shock group; a cardiac arrest group; a pulmonary edema group; a respiratory failure group; a myocarditis group; and an arrhythmogenic right ventricular cardiomyopathy. Each group may consist of some or all of the items in FIG. 3.

The cardiac dysfunction category consists of items where the state of the target patient indicates a disease or state that may be diagnosed as cardiac dysfunction. The cardiac dysfunction category may also be categorized into multiple groups. Each group may also consist of at least one item.

In an embodiment, the cardiac dysfunction category may be categorized into one or more groups of: left ventricular failure with preserved contractility; left ventricular failure without preserved contractility; right ventricular failure; valve dysfunction; cardiomyopathy; pericardial effusion; and/or cardiac compression (or tamponade). Each group may consist of some or all of the items in FIG. 3.

The valve dysfunction group consists of items that each indicate a site-specific symptom. For example, the valve dysfunction group may consist of items that each indicate stenosis and regurgitation symptoms for sites such as the tricuspid valve, bicuspid valve, pulmonary artery, and aorta.

The cardiomyopathy group consists of items that indicate one or more pathologies. For example, the cardiomyopathy group may consist of multiple items that each indicate hypertrophic cardiomyopathy, stress cardiomyopathy, dilated cardiomyopathy, ischemic cardiomyopathy, etc.

In an embodiment, a hereditary arrhythmia category may consist of items of an underlying heart disease that may result in some of the arrhythmia types. The items in the hereditary arrhythmia category indicate various underlying heart diseases that are more likely to cause arrhythmia.

For example, the hereditary arrhythmia category may include one or more items of brugada syndrome, long QT syndrome, catecholaminergic polymorphic ventricular tachycardia, short QT syndrome, early repolarization syndrome, idiopathic ventricular tachycardia, hypertrophic cardiomyopathy, and/or arrhythmogenic right ventricular cardiomyopathy.

In some embodiments, the server 20 may determine whether the target patient has at least one item corresponding to the hereditary arrhythmia category only when the target patient is judged to have an arrhythmia, the type of which does not affect the baseline of the electrocardiogram signal of the target patient. The underlying heart disease item in the hereditary arrhythmia category is associated with only some arrhythmia types among a plurality of pre-specified arrhythmia types. These arrhythmias are described in more detail below.

The other diseases/situations are not included in the categories above, but may be consist of diseases/situations that may be considered in evaluating the state of the patient.

The server 20 may also evaluate the target patient by determining, based on the electrocardiogram image or electrocardiogram signal of the target patient, whether the target patient corresponds to an item in a different aspect from a heart rhythm.

In an embodiment, the server 20 may extract waveform data of the electrocardiogram signal from the electrocardiogram image; and evaluate the target patient based on the extracted waveform data.

The server 20 may include a first artificial neural network pre-trained to extract waveform data of a signal from an input image.

The first artificial neural network is modeled to extract time series data as waveform data according to the number of leads (L) from the input image.

In an embodiment, the first artificial neural network may output a one-dimensional vector of a predetermined length. This length is a preset time interval. In an example, when a hyperparameter N of a time interval is preset for the first artificial neural network, the first artificial neural network calculates a set of one-dimensional vectors (NL1, NL2, NL3, . . . , NLm) for each lead (L1, L2, L3, . . . , Lm).

In an example, the first artificial neural network may be modeled as a structure with multiple output stages. Each output stage corresponds to each lead of the electrocardiogram measuring device.

The first artificial neural network may be modeled as a variety of network structures for classifying whether each pixel of the input image is a waveform of a signal. For example, the first artificial neural network may be modeled as a structure such as 2D-CNN, visual transformer (VIT), multilayer perceptron (MLP), or any other machine learning network structure that includes an improved neural network structure that is extended therefrom (residual connection, max/average pooling, channel attention, spatial attention, layer-normalization, batch normalization, depthwise convolution, bottomleneck, inverted bottomleneck, gating, dropout, or bayesian layer). These neural networks may be applied not only to the first neural network, but also to second and third neural networks.

The waveform data of the electrocardiogram signal calculated from the first artificial neural network is supplied to a different artificial neural network that evaluates the target patient from the input waveform data.

As described above, in an embodiment, the server 20 may supply the electrocardiogram image itself to the artificial neural network evaluating the target patient without the need to extract the waveform data of the signal in the input image from the first artificial neural network.

The server 20 may calculate from the input data an evaluation value representing the likelihood that the target patient corresponds to an item in a different pre-specified aspect. The evaluation value represents the probability that the target patient has a disease of an item, or is a state event. The server 20 may also generate evaluation information including this evaluation value.

In addition, the server 20 may select at least one item based on an evaluation value for each item. The selected item indicates a disease or state event that the target patient is determined (or diagnosed) to have. The server 20 may select an item with the highest degree of correspondence or multiple items with relatively high degrees of correspondence. The server 20 may generate evaluation information including results of these selections.

In an embodiment, the server 20 may include a second artificial neural network pre-trained to calculate the evaluation value of the target patient for a pre-specified item by inputting waveform data and/or a third artificial neural network pre-trained to calculate the evaluation value of the target patient for the pre-specified item by inputting an electrocardiogram image.

The second artificial neural network is modeled to calculate the evaluation value of the target patient for the pre-specified item by processing the input waveform data. The server 20 may input a set of one-dimensional vectors output from the first artificial neural network as input waveform data to the second artificial neural network.

When a set of one-dimensional vectors for each lead is input, the second artificial neural network processes them to calculate the probability that the state of the target patient corresponds to each of the pre-specified items.

The second artificial neural network may be modeled as a variety of network structures for calculating the probability that the state of the target patient corresponds to each evaluation item based on the values of respective one-dimensional vectors in the set. For example, the second artificial neural network may be modeled as a fully connected layer structure or other 1D-CNN structure.

The third artificial neural network is modeled to calculate the evaluation value of the target patient for the pre-specified item directly from the input image by processing an electrocardiogram image in which an electrocardiogram signal is represented as a subregion (e.g., an electrocardiogram signal patch). Instead of extracting the waveform data of the signal from the electrocardiogram image through the first artificial neural network, the server 20 may process the image data through the third artificial neural network to calculate the evaluation value of the target patient.

The third artificial neural network may be modeled as a variety of network structures for processing the input image to calculate the evaluation value for the pre-specified item. For example, the third artificial neural network may be modeled as a structure such as 2D-CNN, visual transformer (VIT), multilayer perceptron (MLP), or any other machine learning network structure that includes an improved neural network structure that is extended therefrom (residual connection, max/average pooling, channel attention, spatial attention, layer-normalization, batch normalization, depthwise convolution, bottleneck, inverted bottleneck, gating, dropout, or bayesian layer).

The second and third artificial neural networks may also perform calculations by applying various activation functions (e.g., sigmoid, Tanh, ReLU, Leaky RELU, Parameterized RELU, ELU, SeLU, GeLU, Swish, and Mish functions) to internal values thereof.

The server 20 may also calculate the evaluation value and/or an item selection result through the second and third artificial neural networks.

Since the second and third artificial neural networks are analytical artificial neural networks that evaluate the state of the target patient, they may have at least partially the same or similar structures and functions.

A form of calculation of the evaluation value depends on output stages of the second and third artificial neural networks.

In an embodiment, at least one artificial neural network of the second artificial neural network or the third artificial neural network may calculate an absolute evaluation value that individually represents the likelihood of each of the pre-specified items. Such an artificial neural network is modeled to have a multi-label output stage. Then, the server 20 independently judges whether the target patient has a disease or state event of each item.

For example, suppose that the second artificial neural network or the third artificial neural network is configured to calculate evaluation values for N pre-specified items through the sigmoid function and softmax function as a computation result from the corresponding artificial neural network. When at least one of the artificial neural networks is configured to calculate an absolute evaluation value, the corresponding artificial neural network may calculate N absolute evaluation values for each of the N items. Each individual absolute evaluation value is calculated as a real number in the range of 0 to 1. The server 20 may provide an independent item evaluation for each of the N items based on the N absolute evaluation values.

In an embodiment, at least one artificial neural network of the second artificial neural network or the third artificial neural network may be configured to calculate a relative evaluation value, which represents a relative likelihood of corresponding to any item within the range of pre-specified items. Such an artificial neural network is modeled to have a multi-class output stage. Each item is treated as a class.

Suppose that the second artificial neural network or the third artificial neural network is configured to calculate evaluation values for N pre-specified items using a probability function such as the softmax function. When at least one of the artificial neural networks is configured to calculate a relative evaluation value, the calculated evaluation value is a real number in the range of 0 to 1 for each item, and the N real numbers have a probability distribution relationship such that the sum of the N real numbers equals 1.

For example, when the artificial neural network calculates the evaluation values for the items in FIG. 3, the total sum of the evaluation values for each item in FIG. 3 equals 1.

Then, the server 20 may select the at least one item as a disease or state event that the target patient has based on the relative evaluation value. For example, the server 20 may select K (where K is a positive number) items that are most likely to be the target patient's heart rhythm among multiple heart rhythm items and calculate the item evaluation for the target patient's heart rhythm. As such, the selection result at least partially reflects the relative item evaluation, expressed as a real number value as described above.

In an embodiment, at least one artificial neural network of the second artificial neural network or the third artificial neural network may be further configured to calculate one or more measurement values associated with the electrocardiogram signal.

The measurement value may include pulse rate, atrial contractions per minute, PR interval, QRS interval, QT interval, a length and interval of corrected QR interval, a QRS axis, a P-axis, and/or a T-axis. In this case, the server 20 may use the calculated value directly as the measurement value, or may use a scaled version of the calculated value as the measurement value.

In addition, the server 20 may be configured to calculate an age of heart from the electrocardiogram signal. The server 20 may also calculate an age of heart that is different from an age with reference to birth through a separate artificial neural network trained to calculate an age of heart.

The server 20 may also be configured to calculate an estimate of a heart calcium score. The server 20 may also calculate the estimate of the heart calcium score based on the degree of calcium deposition in the coronary arteries.

In addition, the server 20 may be configured to calculate an estimate of hemoglobin in the heart.

In alternative embodiments, the second artificial neural network and the third artificial neural network may include multiple artificial neural networks.

The second artificial neural network or the third artificial neural network may include sub-artificial neural networks that each calculate a separate evaluation value. For example, when the server 20 is configured to calculate P evaluation values, there may be P sub-artificial neural networks.

The second artificial neural network or the third artificial neural network may include sub-artificial neural networks that calculate at least two evaluation values. For example, when the server 20 is configured to calculate P evaluation values, there may be Q sub-artificial neural networks (Q<P).

The server 20 may calculate these evaluation values for each item, selection results, and measurement values as the item evaluations.

Additionally, the server 20 may be further configured to calculate distribution information for the result of the evaluation (e.g., an evaluation value) for each item.

In an embodiment, the server 20 may acquire specific distribution formed by an evaluation value for an item at each sequence, which is calculated by repeatedly performing the same evaluation operation multiple times using the same input values.

In an embodiment, the server 20 may acquire specific distribution formed by an evaluation value for an item at each sequence, which is calculated by performing multiple evaluation operations with a transformed input value in which the augmentation technique used in training the second artificial neural network or the third artificial neural network is applied to the same input value multiple times. The augmentation technique may be any transformation method that applies random transformations to the existing image input value within a predetermined intensity.

In an embodiment, the server 20 may input the same input value to different artificial neural networks (e.g., the second artificial neural network or the third artificial neural network) to acquire a specific distribution formed by the evaluation value for an item for each artificial neural network.

The server 20 selects some or all of the aforementioned specific distributions to calculate distribution information of the evaluation value for the item. Any one of the specific distributions may be used directly as the distribution information for the evaluation value, or a combination of a plurality of specific distributions of the evaluation value for the same item may be used as final distribution information for the evaluation value for the item.

The distribution information includes a representative value (e.g., mean, median, etc.) that summarizes the tendency of the distribution, and the degree of distribution (e.g., confidence interval, standard deviation, standard error, interquartile range, and range).

When the distribution is computed or acquired, in addition to acquiring distributions through randomized transformation of inputs (augmentation) and/or acquiring distributions of outputs from multiple networks trained for the same purpose but different from each other, the neural network itself may add randomness to the computational process of processing the input data and transform the computational results to acquire the distribution.

The method for calculating or acquiring the distribution may be to use, for example, 1) a Monte Carlo dropout layer (the dropout layer is also applied in the process of inference) or 2) a Bayesian layer (a method where an output result from a layer is a probability distribution of input values of the next layer, such as mean and standard deviation of Gaussian distribution, and randomly selected output values from this distribution are transmitted to the next layer) included in the neural network, but is not limited thereto, and may also include using all layers for inference in a randomized manner.

The server 20 generates evaluation information including an evaluation value for each item, a selection result, a measurement value, an age of heart, an estimate of a heart calcium score, an estimate of hemoglobin, and/or distribution information on the evaluation value for an item.

In an embodiment, the evaluation information may include one or more of an electrocardiogram image; first diagnostic information; second diagnostic information; and third diagnostic information.

The electrocardiogram image may be an image directly used in the evaluation, for example, a source electrocardiogram image or an electrocardiogram signal patch consisting of an electrocardiogram signal region extracted from the source electrocardiogram image.

The first diagnostic information includes a heart rhythm. The first diagnostic information may include at least one arrhythmia type or a normal rhythm among a plurality of arrhythmia types that the target patient is predicted to have.

The second diagnostic information includes an item (i.e., a disease/state event) and/or an evaluation value of the item. For example, the first diagnostic information includes the evaluation value of the target patient based on the items in FIG. 3.

The third diagnostic information includes one or more of an age of heart, an estimate of a heart calcium score, a measurement value associated with the electrocardiogram signal, and an estimate of hemoglobin.

In addition, the evaluation information may further include an evaluation time, an evaluation date, and/or at least some of the patient's personal information (e.g., a full name).

The evaluation information is provided to a user through the user terminal 10.

Additionally, the plurality of arrhythmia types may be divided into a first group of arrhythmia types and a second group of arrhythmia types.

The first group of arrhythmia types consists of arrhythmia types that correspond to some of a plurality of pre-specified arrhythmia types in the system.

When the target patient has a heart rhythm belonging to the first arrhythmia types, the electrocardiogram signal of the target patient has a waveform that makes it difficult to accurately calculate the likelihood of different heart diseases. This is because the first group of arrhythmia types affect the baseline of the electrocardiogram signal of the target patient. The effect of the first arrhythmia types is always reflected in the baseline of the electrocardiogram signal of the target patient.

In an embodiment, the first group of arrhythmia types may consist of pre-specified arrhythmia types, including atrial flutter, PSVT, atrial tachycardia, ventricular tachycardia, and/or a pacemaker, etc.

Meanwhile, the second group of arrhythmia types is independent of the electrocardiogram signal of the target patient and does not affect the baseline of the electrocardiogram signal of the target patient. The second group of arrhythmia types may consist of some or all of the remaining arrhythmia types that do not belong to the first group among all of the plurality of pre-specified arrhythmia types described above.

When the server 20 determines that the target patient does not have a normal rhythm, the server 20 determines that the target patient has at least one of the plurality of pre-specified arrhythmia types. For example, the server 20 may determine the arrhythmia type of the target patient through the second artificial neural network or the third artificial neural network pre-trained to determine the type of heart rhythm. The determined arrhythmia type belongs to the first group or the second group.

The server 20 may also be configured to evaluate the target patient for an item in a different aspect from the heart rhythm when the heart rhythm of the target patient is determined to be the second group of arrhythmia types.

When the type of heart rhythm of the target patient determined by the server 20 is an arrhythmia type belonging to the second group, the evaluation results in various aspects other than the heart rhythm, as described above with reference to FIG. 3 and the like, may be provided to the user terminal 10. For example, when the server 20 determines one of the second group of arrhythmia types as the heart rhythm of the target patient, the server 20 calculates a result of evaluating whether the heart rhythm corresponds to a category different from the heart rhythm category, as described above, and provides the result to the user terminal 10.

When the type(s) of heart rhythm of the target patient determined by the server 20 includes an arrhythmia type belonging to the first group, among the results of the evaluation in various aspects, evaluation information including only the result of the evaluation in an aspect of heart rhythm may be generated.

When the server 20 determines one of the first group of arrhythmia types as the heart rhythm of the target patient, the server 20 may immediately generate the evaluation information without performing the evaluation of different aspects. That is, the evaluation value is not calculated for an item in a different category from the heart rhythm. The server 20 immediately generates the evaluation information including the result of the evaluation on the heart rhythm of the target patient, or the result of the evaluation on the heart rhythm of the target patient and the electrocardiogram image.

Then, the user terminal 10 may be provided with the evaluation information including the evaluation of the arrhythmia of the target patient determined to be one of the first group of arrhythmia types.

### Reporting Screen

The user terminal 10 may also be configured to receive the evaluation information from the server 20 and display a reporting screen.

FIG. 3 is a view illustrating a reporting screen including an electrocardiogram image and evaluation results, according to an embodiment of the present application.

With reference to FIG. 3, the reporting screen is configured to include one or more of: a first region 1301 displaying an electrocardiogram image; a second region 1310 displaying first diagnostic information; a third region 1320 displaying the first diagnostic information and/or second diagnostic information in a graphical form; and a fourth region 1330 displaying third diagnostic information.

The electrocardiogram image displayed in the first region 1301 is an image used for evaluation (which may be referred to as an evaluation electrocardiogram image compared to a source electrocardiogram image), for example, an electrocardiogram signal patch.

When the type of heart rhythm that the target patient is predicted to have is selected by the server 20, information on the selected type of heart rhythm is displayed on the second region 1310. For example, when a normal rhythm is selected for the target patient, the reporting screen visually displays a text or picture indicating the normal rhythm.

In an embodiment, an item of the first diagnostic information and/or the second diagnostic information and an evaluation value of the item may be displayed graphically on the third region 1320 in the reporting screen. In the third region 1320, the x-axis of the graph represents at least some of the pre-specified items evaluated by the server 20, and the y-axis represents an evaluation value, for example, the evaluation value of the target patient for the corresponding item, such as a probability of a disease/state event.

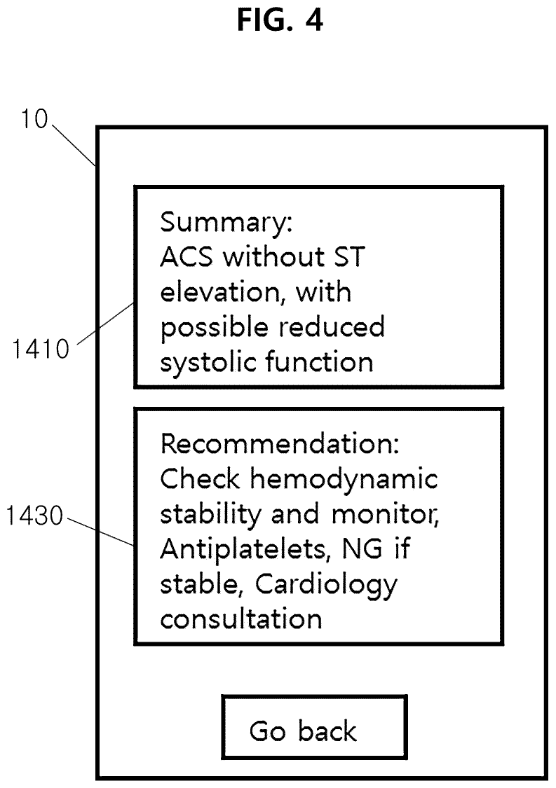

In an embodiment, an evaluation value for each item may be represented as a pointer on the third region 1320. For example, as illustrated in FIG. 4, the evaluation value for each item may be represented as a pointer in red, or may be represented as a pointer in other colors. The user may also be provided with the probability that the target patient has a disease or state event of the corresponding item based on the position of the pointer.

In addition, the second diagnostic information may further include distribution information on the evaluation value for an item.

In an embodiment, the reporting screen may be further configured such that the distribution information of the evaluation value for the item is represented as a sub-region on the third region 1320 in which a pointer of the evaluation value is positioned. For example, as illustrated in FIG. 4, the sub-region that encloses the pointer indicating the evaluation value for each item indicates the distribution information for the corresponding item evaluation.

Detailed information within the distribution information, such as a confidence interval, may also be represented by the shape of the sub-region. For example, the confidence interval may be expressed as a vertical length of the sub-region.

In addition, the reporting screen may be further configured to display a button 1400 that guides a display command for information that summarizes the evaluation information of the target patient. When a command is input to select the displayed button 1400, the user terminal 10 may provide the user with a screen summarizing the evaluation information in response to the input of the command.

In an embodiment, the user terminal 10 may output the evaluation information in the form of a text (or natural language) as a method of providing a screen summarizing the evaluation information. For example, based on the type of arrhythmia and the probability of each disease, when a specific type of arrhythmia is higher than or equal to a specific probability or within a specific ranking, or when the probability of an average value of a specific disease is higher than or equal to a pre-specified value (e.g., J %), or when the probability of being higher than or equal to a pre-specified value (e.g., L %) when integrated based on its distribution is higher than or equal to a pre-specified value (e.g., M %), the evaluation information may be provided to the user by referring to a predefined medical description (pathophysiology, recommended additional tests/first aids (type of medicine, dose, type of treatment, method of treatment/follow-up), etc. may be included) of the corresponding morbidity, for example, a database of pre-stored descriptions.

FIG. 4 is a view illustrating a screen summarizing evaluation information, according to an embodiment of the present application.

With reference to FIG. 4, the summarizing screen includes a region 1410 that relatively summarizes the state of the patient.

In addition, the summarizing screen may further include a region 1420 that displays recommendations for the target patient under the currently evaluated state.

With reference back to FIG. 3, the reporting screen may be further configured to display buttons 1510, 1520, 1530, and 1540 that guide one or more control commands related to the evaluation. These buttons may be physical buttons or virtual buttons implemented as digital images.

In an embodiment, the reporting screen may further include one or more buttons: a button 1510 that guides the initiation of a new evaluation operation; a button 1520 that guides the saving of evaluation information currently expressed on the reporting screen; a button 1530 that guides the input of auxiliary information on the target patient; and a button 1540 that guides the management of the evaluation information on the target patient.

When a command is input to select the displayed eighth button 1530, the user terminal 10 may provide the user with an interface screen for inputting auxiliary information on the target patient in response to the input of the command. The input auxiliary information may be processed as additional input by being concatenated into a vector of intermediate calculation results of N (1 or more) previous fully connected layers from a final output after preprocessing such as one-hot encoding, Z-transformation, and min-max scaling as necessary. Further, by passing this input through a separate artificial neural network that is capable of processing this input, a new output may be created that reflects this information, and a new result of the evaluation that reflects the corresponding output may be output, replacing the existing result of the evaluation.

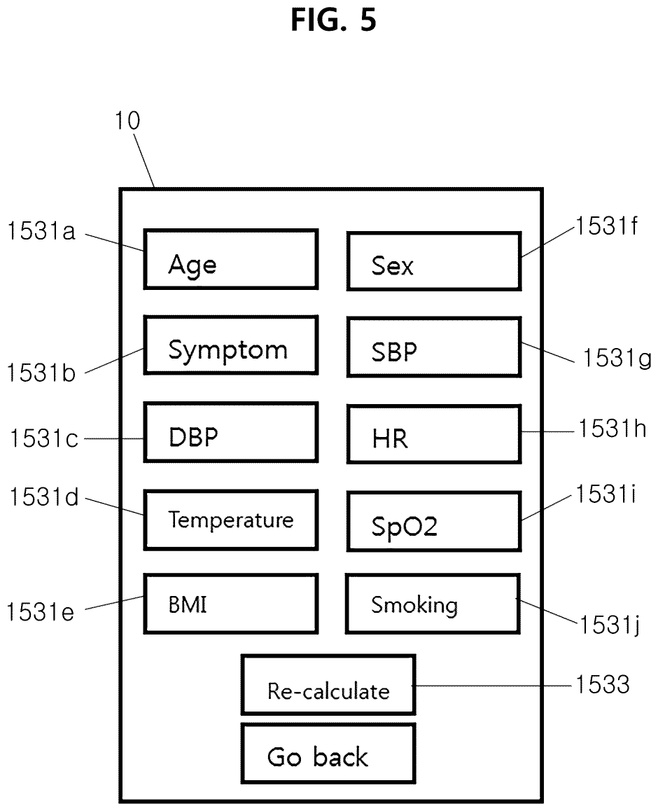

FIG. 5 illustrates an interface screen for inputting auxiliary information on a target patient, according to an embodiment of the present application.

With reference to FIG. 5, the user terminal 10 is configured to receive an input of auxiliary information through the interface screen. The auxiliary information may include the target patient's age, gender, chief complaint, vital sign, oxygen saturation, BMI, and/or smoking status.

The interface screen in FIG. 5 may include input fields 1531 for inputting each auxiliary information.

When receiving the auxiliary information, the user terminal 10 transmits a message requesting to calibrate the evaluation of the target patient based on the auxiliary information to the server 20.

The interface screen in FIG. 5 includes a button 1533 to transmit a calibration request.

When receiving the message, the server 20 may re-evaluate the state of the target patient based on the auxiliary information and generate new evaluation information. For example, the server 20 may recalculate the risk of developing atrial fibrillation in N years based on the auxiliary information. Then, the reporting screen in FIG. 3 is updated with the new evaluation information.

With reference back to FIG. 3, upon inputting a command to select the displayed button 1540, the user terminal 10 may be further configured to display a result of clustering patients with matched evaluation information in response to the input of the command, or to display evaluation information over time for a single patient.

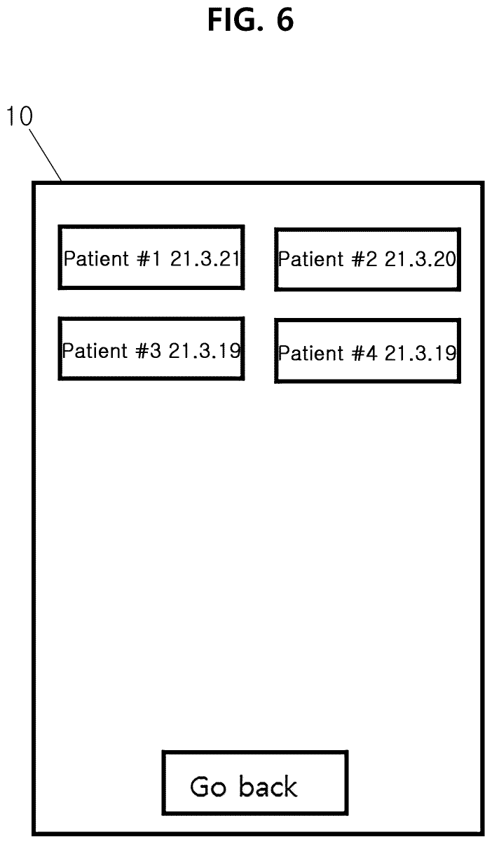

FIG. 6 is a view illustrating a first matching screen displaying a cluster of matching patients, according to an embodiment of the present application.

With reference to FIG. 6, when a command to select the button 1540 is input, the user terminal 10 may transmit a request for clustering the evaluation information of the target patient in the reporting screen in FIG. 3 with other patients to the server 20.

In an embodiment, when receiving the request, the server 20 may search for at least one different patient having evaluation information that matches the evaluation information of the target patient of the request. The server 20 may cluster the searched patient and the target patient into a single cluster.

In some embodiments, the server 20 may pre-store clustering information consisting of patients whose evaluation information matches each other. When receiving the request, the server 20 may search for a cluster having evaluation information that matches the evaluation information of the target patient of the request. The server 20 may update a list of patients in the cluster by adding the target patient to the searched cluster.

The server 20 may reply to the user terminal 10 with the clustering information that includes clustering results. The clustering information may include at least some of personal information of the patient and/or evaluation time.

Then, the user terminal 10 may provide the user with a first matching screen in FIG. 6 that displays patients included in the same cluster.

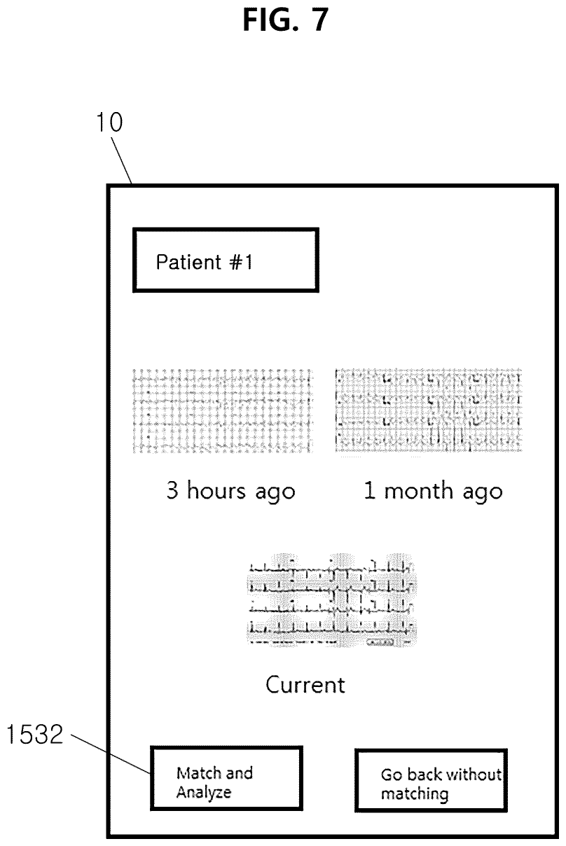

FIG. 7 is a view illustrating a second matching screen for calculating evaluation information on a single patient over time, according to an embodiment of the present application.

With reference to FIG. 7, when a command to select a button 1532 is input, the user terminal 10 may be configured to display a second matching screen that guides a command to select past evaluation information of the target patient to be compared to the evaluation information of the target patient on the reporting screen in FIG. 3.

The second matching screen may be configured to guide a command to select one or more past points in time. At each past point in time, the electrocardiogram image used for evaluation may indicate the corresponding past point in time. Alternatively, the second matching screen may display a phrase describing each past point in time.

In an embodiment, with a method of showing how the current state of the patient has changed from before using electrocardiograms taken at different points in time on the same patient, each electrocardiogram is analyzed in the same way and the results are displayed simultaneously on a single screen so that the user may see what changes have occurred. In particular, when displaying the risk of a disease in a graph, the same item before and after a particular point in time may be displayed together in a single position such that the output is more comparable for the user.

In another embodiment, electrocardiograms taken at different points in time for the same patient may be passed through the same network structure and the output vectors (not necessarily the same N) calculated at the N^{th} layer may be concatenated to create a fusion vector and passed through a separately trained artificial neural network (or any machine learning method that can handle regression and classification with vector inputs, such as Random forest, [Extreme] gradient boost, SVM, Lasso, and Elastic Net). As a result, some (acute myocardial infarction, worsening of extensive heart failure, worsening of pulmonary arterial hypertension, shock/respiratory failure/risk of death) or all of the patient evaluation items already mentioned may be output with increased accuracy in consideration of both electrocardiogram information.

The evaluation information at a selected past point in time in the system 1 is compared to the current evaluation information.

When a command to select a button region 1541 of the second matching screen is input, the user terminal 10 transmits a request for comparing and evaluating the past evaluation information selected on the second matching screen and the current evaluation information to the server 20. The server 20 may calculate an analysis result comparing the current evaluation information to the selected past evaluation information. For example, based on the evaluation value, the server 20 may calculate a comparative analysis result that includes whether the state represented by each item has improved, and the change in the risk of developing atrial fibrillation in N years. The server 20 may reply to the user terminal 10 with the calculated comparative analysis result. Then, the user terminal 10 may provide the comparative analysis result to the user.

It will be apparent to one of ordinary skill in the art that the system 1 for electrocardiogram image-based patient evaluation may include other components not described in the present specification. The system 1 for patient evaluation may also include other hardware elements necessary for the operation described in the present specification, including a network interface and protocol, an input device for data entry, and an output device for printing or other data display.

A method of electrocardiogram image-based patient evaluation (hereinafter “patient evaluation method”) according to another aspect of the present application is performed by at least one computing device including a processor. For example, the patient evaluation method may be performed by the system 1 for electrocardiogram image-based patient evaluation in FIG. 1. Hereinafter, for clarity of description, the patient evaluation method will be described in more detail based on embodiments performed by the system 1 for patient evaluation in FIG. 1.

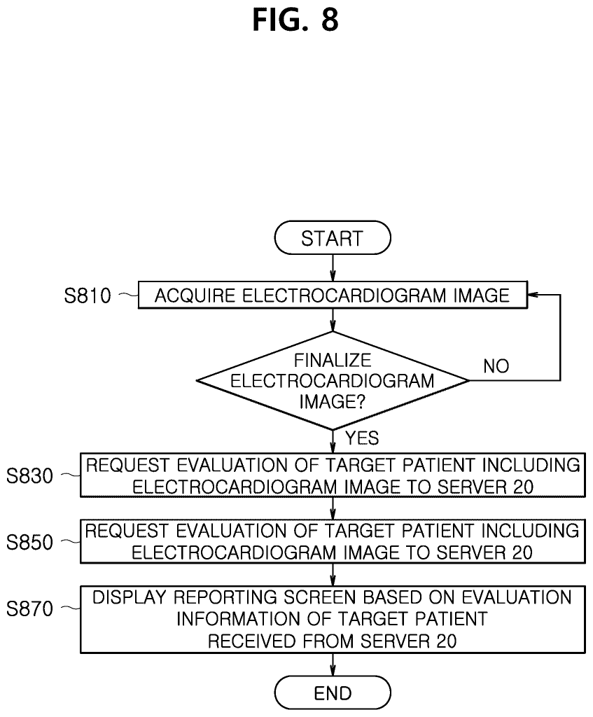

FIG. 8 is a flowchart of a method of electrocardiogram image-based patient evaluation, according to an embodiment of the present application.

With reference to FIG. 8, the patient evaluation method includes a step of acquiring an electrocardiogram image of a target patient by the user terminal 10 (S810).

In an embodiment, the user terminal 10 may be further configured to provide a frame guideline to guide the photographing of a source electrocardiogram image that meets a preset size reference (S810).

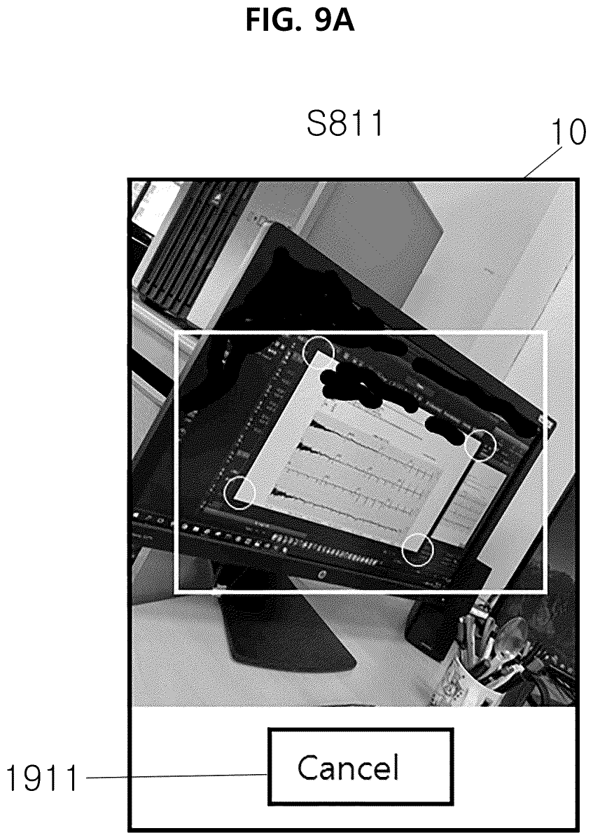

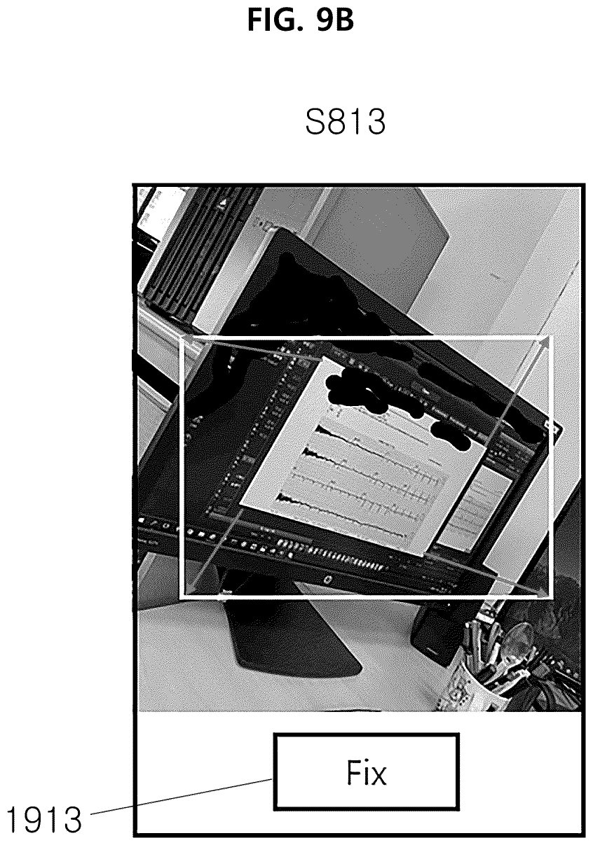

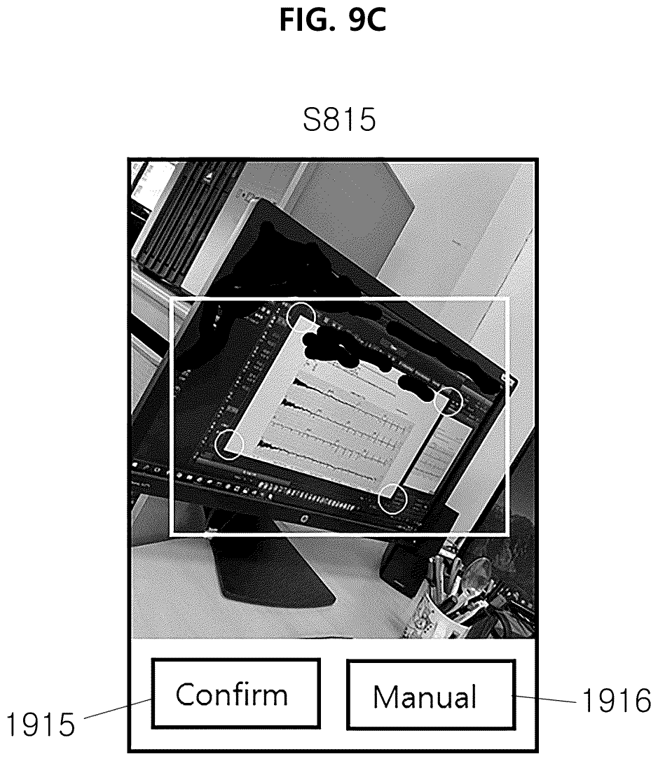

FIGS. 9A to 9C are views illustrating a process of acquiring a source electrocardiogram image according to a frame guideline, according to an embodiment of the present application.

With reference to FIGS. 9A to 9C, the user terminal 10 may edit the photographed image of the target patient according to the frame guideline to generate the source electrocardiogram image. The step of acquiring the electrocardiogram image of the target patient (S810) includes a step of selecting a plurality of reference points for determining a frame guideline in a photographed image (S811), a step of adjusting a size of an electrocardiogram signal region in the photographed image to fit the frame guideline determined in step S811 (S813), and a step of generating a source electrocardiogram image (S815).

In step S811, the photographed image is acquired by the user terminal 10 by photographing an object on which the electrocardiogram signal of the target patient is displayed.

In step S813, the user terminal 10 may select a plurality of reference points in the photographed image to determine a frame guideline for the source electrocardiogram image.

The reference points are geometric feature points that are more useful for defining electrocardiogram signal regions. For example, the reference point may be an edge of a portion including the electrocardiogram signal region.

The selection of the plurality of reference points is performed by a user selection command received through the interface screen in FIG. 9A.

In an embodiment, the plurality of reference points may be input sequentially according to a preset sequence. A position of each reference point is identified in the sequence. For example, the user terminal 10 may identify a first input reference point as an upper left point (LUQ), a second input reference point as an upper right point (RUQ), a third input reference point as a lower right point (RLQ), and a fourth input reference point as a lower left point (LLQ).

In addition, the user terminal 10 may be configured to mark the selected plurality of reference points to distinguish them from unselected points (or regions) on the photographed image. As illustrated in FIG. 9A, the selected reference point may be represented by a shape such as a circle.

The frame guidelines based on the plurality of reference points may also include electrocardiogram signal regions. In addition, at least some of the selected plurality of reference points may be positioned inside the frame guideline. For example, as illustrated in FIG. 9A, the selected reference point may be a different position from an edge of the frame guideline.

In addition, the user terminal 10 may be configured to redetermine the frame guideline after the frame guideline has been primarily determined. The photographed screen in FIG. 9A may also include a button 1911 for canceling and redetermining the frame guideline that has already been determined.

In step S813, the user terminal 10 may adjust the size of the electrocardiogram signal region to be closer to the determined frame guideline. This resizing is performed by a user adjustment command received through the interface screen in FIG. 9B (S813).

The user adjustment command may be implemented as moving a position of at least one of the selected plurality of points. When the adjustment command is input, the user terminal 10 may move a position of a point to be adjusted closer to the determined frame guideline, such that the position of the point to be adjusted is disposed on the frame guideline.

The user adjustment command may also be input in the form of a preset command, which is a different form from the selection command. For example, the user adjustment command may be a drag, but is not limited thereto.

The interface screen in FIG. 9B includes an adjustment end button 1913. An operation to resize the electrocardiogram signal region is completed by a user input selecting the adjustment end button 1913.

This adjustment command makes the size of the electrocardiogram signal region more similar to the size of the frame guideline.

In step S815, the user terminal 10 may also finalize the size of the electrocardiogram signal region. The user terminal 10 generates a source electrocardiogram image consisting of the electrocardiogram signal region of the finalized size. The finalization of this size is performed by a user finalization command received through the interface screen in FIG. 9C (S815). The interface screen in FIG. 9C may be provided after the adjustment operation in FIG. 9B is completed.

The interface screen in FIG. 9C includes an image finalization button 1915. The operation of acquiring the electrocardiogram image S810 is terminated by a user input of selecting the image finalization button 1915.

In addition, the interface screen in FIG. 9C may further include an image re-adjustment button 1916. The operation of step S811 or step S813 may be re-executed by a user input of selecting the image re-adjustment button 1916.

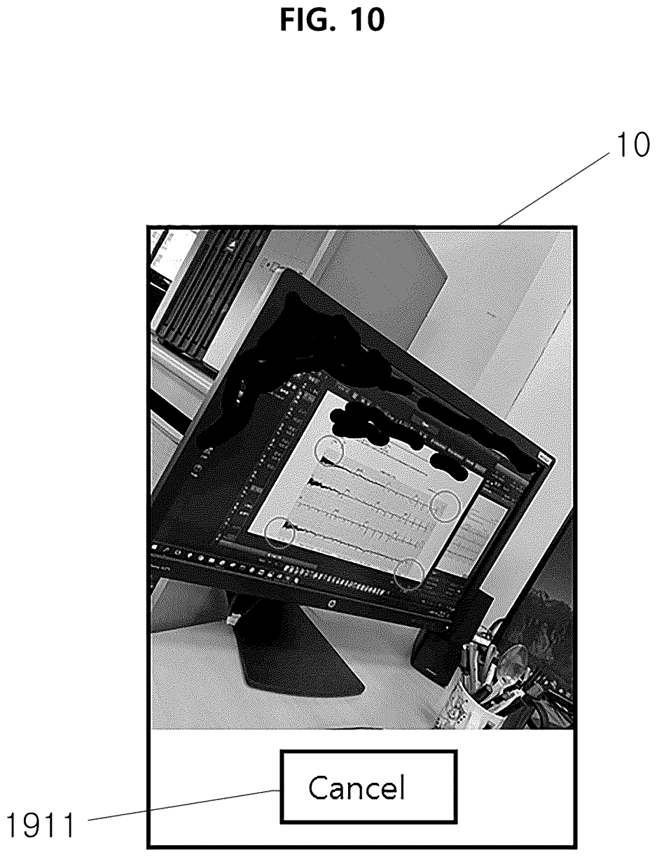

FIG. 10 is a view illustrating a process of acquiring a source electrocardiogram image according to a frame guideline, according to another embodiment of the present application.

With reference to FIG. 10, the reference points may be geometric feature points for distinguishing the electrocardiogram signal regions. For example, an edge of an electrocardiogram signal region may be selected as the reference point.

In alternative embodiments, the acquisition of the electrocardiogram image in the present application is not limited to the frame guideline. The source electrocardiogram image may be acquired through telecommunication from an external device (S810). Alternatively, the source electrocardiogram image may be acquired by searching for a pre-stored electrocardiogram image (S810).

In addition, the patient evaluation method may further include a step S820 of anonymizing the source electrocardiogram image prior to transmitting the source electrocardiogram image of step S810 to the server 20.

The user terminal 10 may be further configured to remove a region that displays the personal information of the patient within the electrocardiogram image of step S810 for anonymization.

In an embodiment, the user terminal 10 may remove the region that displays the personal information of the patient within the source electrocardiogram image by a user input received through the interface screen in FIG. 10.

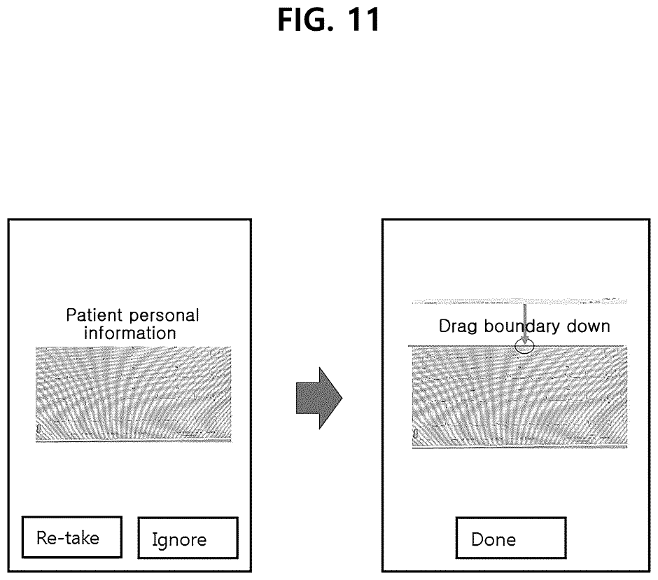

FIG. 11 illustrates an interface screen for anonymizing an electrocardiogram image, according to an embodiment of the present application.

With reference to FIG. 11, a user input is a command to specify a region that displays the personal information of the patient, which may be, for example, a drag, or a click, but is not limited thereto.

In alternative embodiments, the user terminal 10 may remove the region that displays the personal information of the patient within the electrocardiogram image based on some or all of the colors, borders, and disposition structures on the screen, without a user input specifying the region that displays the personal information of the patient.

With reference back to FIG. 8, the patient evaluation method includes a step of requesting evaluation of the target patient by the user terminal 10 to the server 20 (S830).

The request may include the source electrocardiogram image from step S810, and/or the personal information of the patient.

The patient evaluation method includes a step of generating evaluation information of the target patient based on an electrocardiogram image by the server 20 (S850). The electrocardiogram image may be a source electrocardiogram image received from the user terminal 10 or an electrocardiogram signal patch extracted therefrom.

The process of generating the evaluation information of the target patient is described above with reference to FIGS. 2 to 4, and will not be described in detail.

Additionally, the patient evaluation method may further include a step of generating an electrocardiogram signal patch by extracting an electrocardiogram signal region from the source electrocardiogram image prior to step S850 by the server 20.

The server 20 may also perform an operation to generate an electrocardiogram signal patch when an edge of the source electrocardiogram image and an edge of the electrocardiogram signal region do not match.

The server 20 may input the electrocardiogram signal patch as an input image to the artificial neural network (e.g., the first artificial neural network or the second artificial neural network) described above.

In an embodiment, the server 20 may transform the source electrocardiogram image received from the user terminal 10 to a preset size to generate a data array, and apply the transformed image to a fourth artificial neural network to calculate a boundary of a region in which the electrocardiogram signal is represented.

The server 20 transforms the image to a preset size and creates a data array of the transformed image. The data array consists of PXQXC. P and Q are preset horizontal and vertical dimensions, respectively, and C is the number of channels.

The fourth artificial neural network is pre-trained to calculate an upper left coordinate of the electrocardiogram signal region in which the electrocardiogram signal is expressed, a lower left coordinate of the electrocardiogram signal region, an upper right coordinate of the electrocardiogram signal region, and a lower right coordinate of the electrocardiogram signal region. Each coordinate consists of a pair of numbers.

The fourth artificial neural network may be modeled as a network structure for identifying a coordinate of a specific point in the input image. For example, the fourth artificial neural network may be modeled as a 2D-CNN structure or other machine learning network structure.

The server 20 may generate a patch consisting of a region in which the electrocardiogram signal is expressed by cropping the region in which the electrocardiogram signal is expressed in the source electrocardiogram image based on coordinates of four points of the calculated electrocardiogram signal region.

In an embodiment, the server 20 may be configured to process a cropped image with a perspective transformation to generate a transformed image in which at least one of a view, a size, or a shape is transformed, calculate an original aspect ratio of the electrocardiogram signal region in the source electrocardiogram signal, and calibrate an aspect ratio of the transformed image to the original aspect ratio.

The transformed image, for example in the form of a rectangle, is generated by the perspective transformation.

This patch is used as an electrocardiogram image to evaluate the target patient. For example, the electrocardiogram image described above being input to the artificial neural network may also mean that this electrocardiogram signal patch is input.

The server 20 transmits the generated evaluation information of the target patient to the user terminal 10 (S850).

In addition, the patient evaluation method includes a step of displaying a reporting screen based on the evaluation information of the target patient by the user terminal 10 (S870).

For example, as illustrated in FIG. 3, the third region 1320 may be provided with a reporting screen that includes evaluation values for the items, shock, acute coronary syndrome (ACS), acute myocardial infarction requiring emergency procedures such as ST-segment elevation myocardial infarction, any disease accompanied by myocardial damage such as myocardial injury (MYOC INJ), hyperkalemia (SEV HYPERK), hypokalemia (SEV HYPOK), atrial fibrillation (A FIB), various cardiomyopathies, left ventricular failure (Lv Dysf), pericardial effusion (Lrg PC eff), severe mitral regurgitation (Sig mr), and severe pulmonary hypertension (Sig phtn).

The reporting screen has been described above with reference to FIGS. 3 to 7 and will not be described in detail.

According to the system 1 and method for electrocardiogram image-based patient evaluation, a state of a patient can be evaluated in an aspect of heart rhythm, such as arrhythmia, and simultaneously in a different aspect, and the result can be implemented and provided to a user as a reporting screen that is easily recognizable visually by the user.

The operation performed by the system 1 and method for electrocardiogram image-based patient evaluation according to the embodiments described above may be implemented at least in part as a computer program and recorded on a computer-readable recording medium. For example, it may be implemented with a program product configured as a computer-readable medium including program code, which may be executed by a processor to perform any or all of the steps, operations, or processes described.

The computer-readable recording medium includes any kind of recording identification device on which data readable by the computer is stored. Examples of computer-readable storage media include ROM, RAM, CD-ROM, magnetic tape, floppy disks, and optical data storage identification devices. In addition, the computer-readable recording medium may be distributed across a computer system that is networked, so that computer-readable code may be stored and executed in a distributed manner. Further, the functional program, code, and code segment to implement the embodiments will be readily understood by those skilled in the art to which the embodiments belong.

The present application has been described above with reference to the embodiments illustrated in the drawings, which are just for illustration, and those skilled in the art will understand that various modifications and variations of the embodiments are possible. However, such modifications should be considered to be within the technical protection scope of the present application. Accordingly, the true technical protection scope of the present application should be determined by the technical spirit of the appended claims.

### INDUSTRIAL APPLICABILITY

The system for electrocardiogram image-based patient evaluation, according to one aspect of the present application of embodiments of the present invention, can also evaluate a state of a target patient in an aspect of heart rhythm, such as arrhythmia, and in a different aspect, such as another heart disease/state event, based on an electrocardiogram image.

Further, the system for electrocardiogram image-based patient evaluation can also provide a user with a reporting screen configured such that the user can easily recognize visually results of the evaluation in various aspects.

1. A user terminal, comprising a processor configured to evaluate a patient based on an electrocardiogram image,
wherein the user terminal is configured to:
acquire a source electrocardiogram image of a target patient;
transmit a request including the source electrocardiogram image to a server;
receive evaluation information of the target patient from the server; and
provide a reported feedback based on the evaluation information,
wherein the evaluation information comprises one or more of first diagnostic information including an electrocardiogram image and a result of evaluation of a heart rhythm of the target patient, and
wherein the reported feedback comprises one or more of a first region displaying the electrocardiogram image and a second region displaying the first diagnostic information.

2. The user terminal of claim 1, further comprising:
a display unit,
wherein the configuration of providing the reported feedback comprises displaying a reporting screen using the display unit.

3. The user terminal of claim 1, wherein the evaluation information further comprises second diagnostic information including an evaluation value of the target patient for an item in a category different from a heart rhythm category as a result of evaluation in a different aspect from the heart rhythm of the target patient, and
wherein the reporting screen further comprises a third region that displays one or more of the first diagnostic information and second diagnostic information in a graph.

4. The user terminal of claim 3, wherein in the third region, an x-axis of the graph represents at least some of evaluated items and a y-axis represents an evaluation value of the target patient for the corresponding item, and
wherein an evaluation value for each item is expressed as a pointer.

5. The user terminal of claim 4, wherein the evaluation information further comprises distribution information on an evaluation value for an item, and
wherein the reporting screen is further configured such that the distribution information on the evaluation value for the item is expressed as a sub-region on the third region in which a pointer of the evaluation value is positioned therein.

6. The user terminal of claim 5, wherein the sub-region has a length of a shape according to a confidence interval of distribution of each evaluation value.

7. The user terminal of claim 1, wherein the first diagnostic information is calculated by selecting a normal rhythm, any one of a first group of arrhythmia types, or any one of a second group of arrhythmia types, and
wherein when the heart rhythm of the target patient in the first diagnostic information is the second group of arrhythmia types, the user terminal is configured to provide the reported feedback including a result of evaluation consisting only of the electrocardiogram image and the second diagnostic information.

8. The user terminal of claim 7, wherein the first group of arrhythmia types comprises one or more of atrial flutter, PSVT, atrial tachycardia, ventricular tachycardia, and a pacemaker, and
wherein the second group of arrhythmia types comprises some or all of remaining arrhythmias that do not belong to the first group among an entire plurality of pre-specified arrhythmia types.

9. The user terminal of claim 1, further comprising:
a photographing unit,
wherein the user terminal is further configured to:
select a plurality of reference points for determining a frame guideline in a photographed image;
and adjust a size of an electrocardiogram signal region in the photographed image to fit the determined frame guideline, in order to acquire the source electrocardiogram image.

10. The user terminal of claim 9, wherein the determined frame guideline comprises an electrocardiogram signal region, and
wherein at least some of the selected plurality of reference points are positioned inside the frame guideline.

11. The user terminal of claim 9, wherein the user terminal is further configured to remove a region displaying personal information of a patient to anonymize the source electrocardiogram image.

12. The user terminal of claim 1, wherein the user terminal provides a result of evaluation to an expert account registered in a database.

13. The user terminal of claim 12, wherein feedback from the expert account on the result of the evaluation is provided to the user terminal.

14. The user terminal of claim 12, wherein the result of the evaluation provided to the expert account is provided in a manner including at least one of a text message, an email, or a push notification.

15. The user terminal of claim 13, wherein the feedback from the expert account provided to the user terminal is in the form of at least one of an image, a voice, or a text.

16. The user terminal of claim 1, wherein the user terminal provides a result of evaluation to a medical professional or hospital account registered in a database.

17. The user terminal of claim 16, wherein the result of the evaluation provided to the medical professional or hospital account is provided in a manner including at least one of a text message, an email, or a push notification.

18. The user terminal of claim 16, wherein the user terminal further provides the medical professional or hospital account registered in the database with a list of preparations based on the result of the evaluation.

19. The user terminal of any one of claim 16, wherein feedback from the medical professional or hospital account on the result of the evaluation is received and provided to the user terminal.

20. The user terminal of claim 19, wherein the feedback from the medical professional or hospital account provided to the user terminal is in the form of at least one of an image, a voice, or a text.

21-40. (canceled)
